(19) United States
(12) Patent Application Publication
(10) Pub. No.: US 2026/0034251 A1
(43) Pub. Date: Feb. 5, 2026

(54) Radiotracer Labeling and Administration Methods and System

(71) Applicant: Sherice Marion Malagon-King, Denham Springs, LA (US)

(72) Inventor: Sherice Marion Malagon-King, Denham Springs, LA (US)

(21) Appl. No.: 19/355,886

(22) Filed: Oct. 10, 2025

Related U.S. Application Data
(63) Continuation-in-part of application No. PCT/US2023/034104, filed on Sep. 29, 2023.
(60) Provisional application No. 63/764,191, filed on Feb. 27, 2025.
(60) Provisional application No. 63/847,333, filed on Jul. 20, 2025.

(51) Int. Cl. A61K 51/02

(52) U.S. Cl. CPC A61K 51/02; A61K 2123/00

(57) ABSTRACT

Methods are disclosed for electronically labeling and systemically administering radiotracers for targeted diagnostic and therapeutic applications, including immunomodulation. Radiotracers are electronically radiolabeled via direct application to a conductive conductor and delivered systemically through trans-vascular absorption, enabling controlled biodistribution confined to the conductor contact and minimizing off-target exposure. Real-time imaging permits monitoring of radiotracer distribution and activity during administration. Applications include immune modulation for suppression, ablation, or stimulation, as well as activation of non-radioactive precursors for diagnostic and therapeutic use. These methods are applicable to cancer, infectious disease, and autoimmune disorder treatment. Parameters such as radiotracer choice, radioactive activity, precursor formulation, dosing schedule, and administration duration may be adjusted individually or in combination to achieve the desired outcome. These techniques provide reproducible, controllable radiotracer delivery suitable for both imaging and therapy with integrated monitoring.

### CROSS-REFERENCE TO RELATED APPLICATIONS

This patent application claims the benefit of U.S. Prov. Ser. No. 63/764,191 filed Feb. 27, 2025. This patent application claims the benefit of U.S. Prov. Ser. No. 63/847,333 filed Jul. 20, 2025. This patent application claims the benefit of PCT application US2023/034104 filed Sep. 29, 2023. The above applications are incorporated by reference herein.

### FIELD OF THE INVENTION

The present invention relates to the field of medical diagnostics and therapeutic methods in the body, particularly methods of labeling and administering radiotracers for systemic use in disease detection, treatment, and patient management. More specifically, it encompasses novel approaches to radiotracer labeling and delivery for applications in immune modulation, including suppression, stimulation/activation, and ablation, as well as in diagnostic imaging and therapeutic monitoring.

### BACKGROUND

Radiotracers (radiopharmaceuticals) are widely used in human medicine for diagnostic imaging and targeted therapy. Current methods for labeling radiotracers include chemical and non-chemical techniques. Chemical radiolabeling methods rely on creating covalent chemical bonds between radionuclides and a targeting molecule. Common approaches of chemical radiolabeling include electrochemical labeling, direct labeling, and indirect labeling. Electrochemical labeling attaches a radioactive isotope to a non-radioactive molecule using electric current to drive the chemical reaction, creating a radiolabeled compound. Direct radiolabeling directly incorporates the radionuclide into the molecular structure by chemical reaction, such as reduction or adsorption to form a stable bond between the radionuclide and the target. Indirect radiolabeling uses bifunctional chelators to link the radionuclide to a biomolecule without altering its targeting properties. Non-chemical radiolabeling do not involve forming covalent bond but instead rely on physical forces to incorporate radionuclides, and are particularly useful for labeling nanoparticles or liposomes. Common approaches of non-chemical radiolabeling include chemisorption, doping, remote loading, neutron activation, and isotopic exchange. Chemisorption attaches a radioactive isotope to a material's surface by forming a strong, irreversible chemical bond. Doping intentionally incorporates a radionuclide directly into the crystal lattice of a nanoparticle during synthesis to create a highly stable and efficient radiolabeled material. Remote loading encapsulates a radioactive substance into the core of a pre-formed vesicle, such as a liposome, by using an ion or pH gradient across the membrane. Neutron activation bombards a stable, non-radioactive material with neutrons to create an unstable, radioactive isotope that can be used as a tracer. Isotopic exchange is a chemical or physical process where a stable, non-radioactive atom in a molecule is replaced by a radioactive isotope of the same element. These methods, while effective, pose challenges for controlling the radiotracer's systemic distribution and retention in-vivo to minimize off-target radiation exposure based on current administration methods.

Current methods for administering radiotracers include intravenous injection or infusion, oral ingestion, and inhalation. While effective for delivering radiotracers into the body, these routes present several shortcomings for controlled systemic biodistribution. Intravenous and infusion delivery does not promote controlled systemic biodistribution precisely because the radiotracer is subject to the body's natural processes of circulation and biological clearance. The resulting biodistribution is the sum of the radiotracer's initial rapid distribution via the bloodstream and its subsequent, and often slow, elimination by organs. Oral ingestion is subject to metabolic variability and unpredictable absorption, limiting dose control and reproducibility. Standard inhalation methods provide limited systemic exposure and are generally confined to pulmonary distribution.

Existing methods for activating precursors and probes include photoactivation, ultrasound-triggered release, magnetothermal activation, electrochemical or electrode-mediated activation, enzymatic or chemical conversion of prodrugs, and conventional pre-administration radiolabeling. These approaches each represent distinct strategies for controlling when and where therapeutic agents become active in the body. Photoactivation relies on specific wavelengths of light to activate compounds at targeted sites, while ultrasound-triggered release uses acoustic energy to disrupt carriers or alter tissue permeability for localized drug delivery. Magnetothermal activation leverages magnetic fields to heat nanoparticles or carriers, triggering release or conversion of therapeutics. Electrochemical or electrode-mediated activation uses localized electrical potentials to drive redox reactions, liberating or activating drugs in situ. Enzymatic or chemical conversion of prodrugs depends on endogenous or exogenous catalysts to metabolize inactive compounds into pharmacologically active forms, whereas conventional pre-administration delivers active agents systemically without spatiotemporal control. In contrast, trans-vascular absorption of electronically radiolabeled radiotracer(s) enables systemic activation of otherwise non-radioactive precursors in vivo by using energy conversion at the molecular interface, providing a fundamentally different mechanism of distributed, controllable activation throughout the body—with the added advantage of being switched on or off externally, unlike conventional activation methods.

Conventional radiolabeling, administration, and precursor activation limitations also hinder the broader use of radiotracers for systemic immunomodulation. Applications such as immune suppression (e.g., autoimmune disease, graft rejection), immune ablation (e.g., pre-transplant conditioning), and immune stimulation (e.g., cancer or infectious disease therapy) require controlled, transient systemic biodistribution of radiotracers that current methods cannot provide.

Techniques such as conventional external beam radiation (EBRT) and total body irradiation (TBI) are both limited by their external delivery approach in immunomodulation. EBRT is inherently localized, delivering high-energy ionizing radiation to a defined treatment field while minimizing exposure to surrounding healthy tissue. Although EBRT can occasionally produce systemic immune effects, such as the abscopal effect, its primary action is confined to the irradiated site; distant metastases or systemic disease are largely unaffected. TBI, by contrast, delivers radiation externally across the entire body, producing systemic immunosuppression or ablation, and is commonly employed in conditioning regimens prior to hematopoietic stem cell transplantation. TBI, however, is limited in its precision; its external delivery does not allow fine-tuning of dose at the cellular or vascular level. In distinction, trans-vascular absorption of electronically radiolabeled radiotracer(s) provides internal systemic delivery, distributing ionizing energy throughout the vascular system and surrounding tissues. Unlike EBRT and TBI, the dose is administered via the bloodstream rather than as external beams, enabling selective modulation of immune activity at the cellular and tissue level for controlled systemic immune stimulation, suppression, or ablation.

Accordingly, there remains a need for improved methods of radiotracer labeling and administration that overcome the shortcomings of current techniques to expand both diagnostic and therapeutic applications. As studies have explored alternative routes, none of these methods disclose or suggest:
electronically radiolabeling radiotracer(s) via direct contact with a conductor, in which radiotracer energy flows through the conductor to induce an activated radiolabeled state for systemic biodistribution;
controlled systemic biodistribution of electronically radiolabeled radiotracer(s) via trans-vascular contact;
conductor-based trans-vascular absorption, where the conductor is applied to a vascular structure or a surface overlying a vascular structure to enable systemic biodistribution of electronically radiolabeled radiotracer(s), without intravenous injection, and ceases biodistribution of the electronically radiolabeled radiotracer(s) upon the conductor not contacting;
activating a non-radioactive precursor in vivo via conductor-based trans-vascular absorption and systemic biodistribution of electronically radiolabeled radiotracer(s), without intravenous injection, and ceases biodistribution of the electronically radiolabeled radiotracer(s) and activation of the precursor upon the conductor not contacting;
systemic immunomodulation—including suppression, ablation, or stimulation—via trans-vascular absorption of electronically radiolabeled radiotracers; or
real-time monitoring of electronically radiolabeled radiotracer(s) biodistribution and diagnostic and therapeutic effects using imaging modalities sensitive to radiotracer emissions.

Current methods also do not teach or suggest the claimed method of electronic radiolabeling of radiotracers that permits the immiscible labeling of two or more radiotracers for administration via trans-vascular absorption. This approach provides precise control and on-demand flexibility to interchange radiotracers, ensuring rapid and reproducible customized doses for systemic biodistribution. As a result, electronically radiolabeled radiotracer(s) expand potential applications, enabling both diagnostic imaging and therapeutic interventions with greater flexibility and efficiency compared to traditional radiolabeling methods.

Moreover, as electronically radiolabeled radiotracer(s) are administered trans-vascularly, the method allows for multiple administrations over time without the limitations associated with conventional administration routes. Repeated trans-vascular delivery of electronically labeled radiotracer(s) provides flexibility for longitudinal monitoring, sequential therapy, or combination treatment regimens, expanding the range of clinical applications beyond what is possible with single-dose conventional radiopharmaceuticals.

Accordingly, the present invention provides methods using a conductor-based system that establish novel, non-obvious, and clinically versatile approaches to radiotracer labeling and administration. These methods overcome the limitations of current radiolabeling, precursor activation, conventional injection, oral ingestion, and inhalation of radiotracers, and external radiation techniques by enabling controllable, trans-vascular, systemic delivery for reproducible diagnostic and therapeutic applications.

### BRIEF SUMMARY OF THE INVENTION

The invention provides methods for labeling and administering radiotracer(s) for diagnostic and curative functions, using a conductor as a radiotracer administration system to enable controlled delivery and biodistribution of radiotracer(s) in the body.

The methods include:
using a conductor configured for contact with a vascular structure or a surface overlying a vascular structure and electronic radiolabeling of radiotracer(s) by providing a conductive material to facilitate trans-vascular absorption and systemic biodistribution (see FIGS. 1-2);
radiolabeling electronically the radiotracer(s) by directly applying the radiotracer(s) to a conductor made of conductive material to induce activation for systemic delivery (see FIGS. 4);
immiscible electronic radiolabeling, wherein radiotracers are electronically radiolabeled through charge transfer interactions without chemical conjugation (see FIG. 4);
delivering the electronically radiolabeled radiotracer(s) by absorption for systemic biodistribution of radiotracer energy to suppress, ablate, or stimulate immune cells as required for therapeutic effect (see FIGS. 1-3);
delivering the electronically radiolabeled radiotracer(s) by absorption for systemic biodistribution to activate a non-radioactive precursor in vivo for diagnostic and therapeutic purposes (see FIGS. 3);
controlling systemic localization of the electronically radiolabeled radiotracer(s) to occur only during active administration, with dissipation upon cessation to minimize off-target radiation exposure (see FIGS. 1-3); and
monitoring and capturing real-time biodistribution, diagnostic, and therapeutic effects using imaging modalities sensitive to radiotracer emissions during administration (see FIGS. 1-3).

Administration dosing of the electronically radiolabeled radiotracer(s) may be determined based on radiotracer activity, dose rate, dose rate half-life, or dose time. Illustrative embodiments of these methods are shown in FIGS. 1-3 and further described in the Detailed Description.

### BRIEF DESCRIPTION OF THE FIGURES

The accompanying drawings, which are incorporated herein and form a part of the specification, illustrate some, but not the only or exclusive, examples of embodiments and/or features.

FIG. 1 illustrates a method of labeling and administering a radiotracer by trans-vascular contact, including housing a conductive wire within a blunt tip needle, directly applying the radiotracer(s) to the conductive wire (conductor) by connecting (interlocking) a syringe hosting the radiotracer(s) to the conductor-blunt tip needle, electronically radiolabeling the radiotracer(s) upon application to the conductor, and contacting the distal end of the conductor to a vascular structure or a surface overlying a vascular structure to achieve systemic biodistribution of the electronically radiolabeled radiotracer(s).

FIG. 2 illustrates a method of labeling and administering a radiotracer by surface vascular contact, including applying the radiotracer(s) to absorbent pad(s) before direct adhesion to a conductive adhesive tape, electronically radiolabeling the radiotracer(s) upon application to the conductive adhesive tape (conductor), and adhering the conductor to a vascular structure or a surface overlying a vascular structure to achieve systemic biodistribution of the electronically radiolabeled radiotracer(s).

FIG. 3. illustrates a method of activating a non-radioactive precursor via trans-vascular administration of electronically radiolabeled radiotracer(s).

FIG. 4 illustrates a method of electronic radiolabeling and immiscible electronic radiolabeling of radiotracer(s).

With respect to the above description, before explaining preferred embodiments of the herein disclosed invention in detail, it is to be understood that the invention is not limited in its application to the particular steps, sequences, or examples set forth in the following description or illustrated in the drawings. It is also to be understood that the phraseology and terminology employed herein are for the purpose of description and should not be regarded as limiting.

As applicable, herein in the description and claims, the following terms are to be understood as follows:
the term “comprising” refers to methods including but not limited to the listed steps, with additional steps optionally present;
the term “consisting of” refers to methods limited only to the listed steps, with no additional steps present;
the term “consisting essentially of” refers to methods that include the listed steps and may include other steps, provided such other steps do not materially affect the intended function or outcome of the method.
   Unless otherwise specified, all method embodiments described herein are intended to be interpreted in an open and inclusive sense under “comprising.”

### DETAILED DESCRIPTION OF THE INVENTION

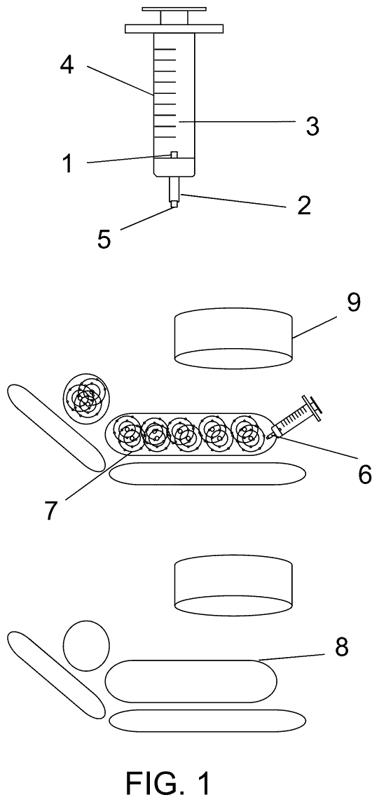

The invention provides methods of labeling and administering radiotracer(s) using conductors made of conductive materials, wherein radiotracers are electronically radiolabeled by direct application to the conductor. The electronically radiolabeled radiotracer(s), via the conductor, is then administered via contact with a vascular structure or surface overlying a vascular structure, enabling systemic biodistribution of the electronically radiolabeled radiotracers for therapeutic or diagnostic applications (FIGS. 1-3).

Radiotracers suitable for these methods include radioligands and radioisotopes, which may be in solid, liquid, or gas form. Radioligands are molecules labeled with a radioisotope and designed with specific binding affinity to targeted cells, tissues, or organs. Radioisotopes are chemical elements produced naturally or synthetically. They may exhibit selective binding, as with radioligands, or non-selective distribution, interacting broadly with cells, tissues, and organs. Radiotracer selection is based on diagnostic or therapeutic intent, and the radiotracer activity (measured in MBq or mCi) and dose rate (measured in Gy or rad) are selected according to the desired exposure.

Radiotracer activity refers to the measure of radioactive decay events per unit time of a radiolabeled compound, typically expressed in units of megabecquerels (MBq) or millicuries (mCi). One megabecquerel (1 MBq) corresponds to 1×10⁶ nuclear decays per second, and one millicurie (1 mCi) corresponds to 3.7×10⁷ nuclear decays per second. These units quantify the absolute radioactive content of a radiotracer sample and are critical in corresponding the dose rate at the radiotracer activity level.

Radiotracers are commonly classified according to the energy of the emitted radiation, which can affect tissue penetration and biological impact. Low-energy radiotracers typically emit beta or gamma radiation with mean energy up to approximately 200 kiloelectron volts (keV). Examples include Technetium-99m (Tc-99m) and Iodine-123 (1-123). Medium-energy radiotracers emit radiation in the range of approximately 200-500 keV and include radioisotopes such as Iodine-131 (1-131) and Lutetium-177 (Lu-177). High-energy radiotracers emit radiation above approximately 500 keV, with greater tissue penetration and cytotoxic potential, including isotopes such as Yttrium-90 (Y-90) and Strontium-89 (Sr-89). Some radiotracers may be classified as medium-to-high energy such as Fluorine-18 (F-18) due to its annihilation photon energy production that results in two 511 keV gamma photons.

The dose rate of the dose is typically measured in milliroentgen (mR) or Gray (Gy) per unit time, representing the amount of energy deposited in tissue per unit mass. The dose rate may depend on the activity of the radiotracer(s). As the radiotracer(s) decays—the process in which a radiotracer losses energy and decreases in activity, in singularity or as a combined dose—as in immiscible labeling, the dose rate may or may not decrease. If the dose rate decreases as the radioactivity decreases, then the dose rate half-life is factored in dose time (administration time) calculations. The dose rate of the dose may be measured using a Geiger counter in mR/hr, then converted to Gy/hr by the multiplication factor of 8.77×10⁻⁶, as in dry air, 1 milliroentgen per hour is equivalent to 0.00000877 Gy. A Geiger counter is typically used to measure dose rates (radiotracer emissions).

When the dose rate does decrease as the radiotracer activity decreases, the dose rate half-life over the radiotracer activity amount in which the dose is administered, must be determined before calculating the dose time. The mR/hr and mCi amount of the dose are measured in corresponding time intervals (e.g. hourly) using a Geiger counter and dose calibrator, respectively. A dose calibrator is typically used to measure radioactivity of radiotracer doses. The mR/hr readings (y-axis) and time interval (x-axis) are plotted on a semi-logarithmic graph (log Y vs. linear X) to determine the absolute value of the dose rate slope. The slope is used to calculate the Dose Rate T½ as:

$$Dose Rate T 1 / 2 = ln ( 2 ) / Slope$$

Where:
Dose Rate T½ is the half-life of the dose rate of the dose in hours;

$$ln ( 2 ) is the natural log of 2 ( = 0.6 \; 93 ) ;$$

Slope is the absolute value of the semi-log Y graph slope.
   This equation is derived from the exponential decay relationship of radiotracer activity, where the decrease in dose rate over time follows a logarithmic curve. By plotting measured dose rate values (e.g. mR/hr) against time on a semi-logarithmic graph (logarithmic Y-axis vs. linear X-axis), the slope of the resulting line corresponds to the decay constant of the dose rate. Since the half-life of a decaying parameter is equal to the natural logarithm of 2 (0.693) divided by the decay constant, the slope of the semi-log plot provides the necessary constant to calculate the dose rate half-life. Thus, the formula directly relates the observed slope of the semi-logarithmic decay plot to the time required for the dose rate to decrease by one-half. Thereafter, the T½ Factor is calculated:

$$T 1 / 2 Factor = ( Dose Amount \times ( ln ( 2 ) / Dose Rate T 1 / 2 ) ) / Dose Rate$$

Where:
T½ Factor is a factor used in calculating the dose time when the slope is not zero and the T½ Factor is less than 1;
Dose Amount is the desired dose to be administered in Gy;

$$ln ( 2 ) is the natural log of 2 ( = 0.6 \; 93 ) ;$$

Dose Rate T½ is the half-life of the dose rate of the dose in hours;
Dose Rate is the dose rate reading of the Dose Amount in Gy/hr.
   This factor represents the ratio between the desired cumulative dose and the maximum deliverable dose when the dose rate is decreasing exponentially due to radioactive decay. It is calculated using the desired Dose Amount (in Gy), the measured Dose rate (in Gy/hr), and the decay constant expressed as ln(2)/Dose Rate T½. The T½ Factor provides a check on whether the target dose can be delivered within the limits of the decaying dose rate. When the value is less than 1 and the slope is not zero, the electronically radiolabeled radiotracer(s) is physically capable of delivering the intended Dose Amount, and the decay-corrected formula for Dose Time T½ Factored must be used to determine the correct administration time. If the T½ Factor is equal to or greater than 1, or the slope is zero, the decay-corrected formula is not applicable, and a simpler constant-rate formula (Dose Time) is used instead. The T½ Factor is therefore necessary in the Dose Time T½ Factored formula because it adjusts the time calculation to account for the decreasing dose rate during administration. Without incorporating this factor, the calculated dose time would be inaccurate, as it would assume a constant dose rate rather than one that diminishes over the course of delivery. If the T½ Factor is less than 1 and the slope is not zero, use the Dose Time T½ Factored formula to calculate the dose time:

$$Dose Time T 1 / 2 Factored = \text{} - ( 1 / ( ln ( 2 ) / T 1 / 2 ) ) \times Ln ( 1 - Dose Amount \times ( ln ( 2 ) / T 1 / 2 ) / Dose Rate ) )$$

Where:
Dose Time T½ Factored is the formula to calculate the dose time (in hours) when the T½ Factor is less than 1 and the slope is not zero;

$$ln ( 2 ) / T 1 / 2 = decay constant ( T 1 / 2 is the Dose Rate T 1 / 2 in hours ) ;$$

Dose Amount is the desired dose to be administered in Gy;
Dose Rate is the dose rate reading of the Dose Amount in Gy/hr.
   This formula is derived by integrating the exponentially decreasing dose rate over time and solving for the administration interval required to achieve the target Dose Amount. The negative sign ensures a positive solution for time, since the logarithm term is less than one. The Dose Time T½ Factored formula is only applied when the slope of the semi-logarithmic plot is non-zero (indicating that the dose rate decreases with decay) and the T½ Factor is less than one (indicating that the target dose can be delivered under the decaying conditions). If the T½ Factor is not less than 1 or the slope is zero, whereas the dose rate does not notably decrease as the radiotracer activity decreases, the dose time is calculated using the simpler constant-rate formula as:

Dose Time=Dose Amount/Dose Rate
Where:
Dose time is the total time the dose is administered;
Dose Amount is the desired dose to be administered in Gy;
Dose Rate is the dose rate reading of the Dose Amount in Gy/hr.
   This formula represents the limiting case where decay correction is unnecessary and is applied whenever the dose rate remains effectively constant as the electronically radiolabeled radiotracer(s) decays during the administration interval or when the T½ Factor is not less than one.

The equations/formulas disclosed herein may be rearranged as necessary to calculate any of the relevant variables. The time units applied in the formulas is not limited to hours, and may be expressed in seconds, minutes, or any other convenient unit, provided that such units are applied consistently throughout the calculations. The dose units applied in the formulas is not limited to Gy, and may be expressed in rad or any other convenient unit, provided that such units are applicable and applied consistently throughout the calculations. Furthermore, the total administered dose, whether defined by dose time or amount, may be fractionated and delivered over multiple discrete periods, such that the intended cumulative dose is achieved through separate administrations rather than a single continuous exposure. The frequency of dose administration may be adjusted as desired, including daily, weekly, or other intervals, to achieve the intended cumulative dose and/or desired biological effect.

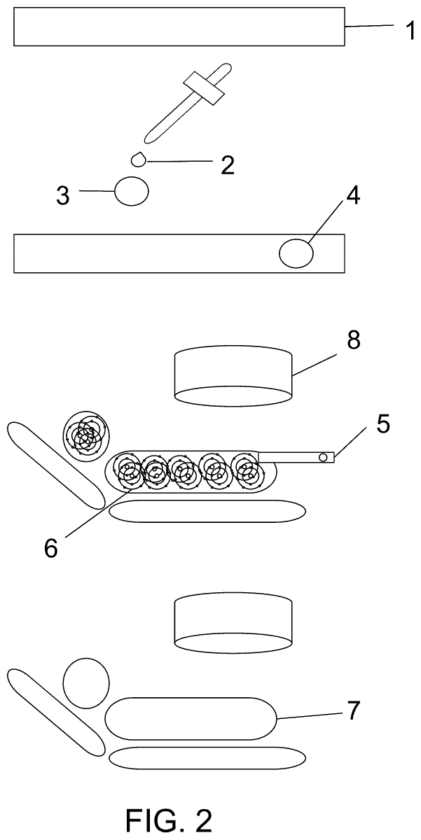

Conductors are fabricated from conductive materials such as, but not limited to, copper, silver, gold, carbon-based composites, or conductive polymers. They may take the form of wires, meshes, foils, or adhesive conductive films or tapes. The conductor geometry enables flow of radiotracer-associated charge and facilitates electronic radiolabeling. Examples include:
wires for trans-vascular absorption (FIG. 1);
adhesive conductive films for surface vascular contact (FIG. 2);

Conductors may be pre-sterilized and housed in modular casings that act as radiation shields, reducing exposure to the contained radiotracer. In some embodiments, casings shield all system components while allowing selective unshielding of conductor parts for administration.

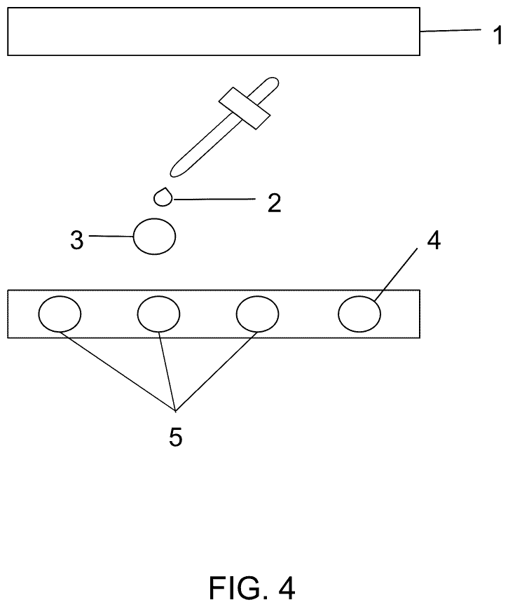

Electronic radiolabeling occurs when the radiotracer is applied to the conductor (FIG. 4), allowing the radiotracer's energy to flow as electric current through the conductive material. This electric current functions as the operative mechanism for trans-vascular transfer of the electronically radiolabeled radiotracer(s). The charged particles emitted from the radiotracer(s) as it decays are captured and directed across the conductive substrate, wherein the conductive element channels the resulting ionizing energy into an electric current. Said current is applied at the site of contact with the vascular microenvironment, where the vascular endothelium exhibits high permeability to ionic and electronic charge exchange. As a result, the current facilitates trans-vascular absorption of the electronically radiolabeled radiotracer(s) across the endothelial barrier. Once transferred into circulation, the electronically radiolabeled radiotracer(s) undergoes systemic biodistribution in accordance with blood flow kinetics, thereby functionally replicating intravenous administration without requiring needle-based vascular access.

Immiscible labeling of radiotracer(s) during electronic radiolabeling is permitted. Immiscible labeling is a method in which two or more radiotracer compounds are electronically radiolabeled (FIG. 4) or activated in such a way that each maintains a distinct, non-mixing phase or chemical environment during the labeling process. This allows multiple radiotracers to be prepared or delivered simultaneously without chemical interference or cross-contamination, preserving the individual radiochemical integrity, activity, and targeting properties of each tracer. Immiscible labeling can be applied to enhance multi-target imaging, combinational therapeutic strategies, or simultaneous diagnostic and therapeutic (theranostic) applications.

In certain embodiments, electronic radiolabeling induces radiolysis of the radiotracer, liberating free radionuclides that are administered for systemic biodistribution. Once circulating in the body, these free radionuclides generate cascades of reactive oxygen species (ROS), which exert direct cytotoxic effects on malignant or virally infected cells. ROS also serve as danger signals, activating innate and adaptive immune pathways, including natural killer (NK) cells and cytotoxic T lymphocytes. This dual-action therapeutic effect combines direct oxidative and radiative destruction of diseased cells with immune activation that enhances recognition and clearance of residual disease. Such radiolytic activation and controlled ROS-mediated immune effects are not achievable with conventional injection, ingestion, or standard inhalation methods, which deliver intact radiotracers that rely on slow biological clearance, leading to prolonged and uncontrolled systemic exposure.

The route of administration is trans-vascular absorption.

### Trans-Vascular Absorption (FIG. 1-3):

a conductor may be configured as a blunt-tip wire or adhesive conductive film;
the conductor is brought into contact with a vascular structure (e.g., superficial vein, arterial wall) or surface overlying a vascular structure (e.g. skin, mucosa);
contact enables radiotracer absorption across the vascular endothelium, resulting in systemic biodistribution without needle injection;
multiple administrations over time are possible.

Multiple administrations of electronically radiolabeled radiotracer(s) over time are enabled by trans-vascular absorption and immiscible labeling. The conductor hosting the electronically radiolabeled radiotracer(s) may be applied sequentially to a single or to multiple bodies, allowing repeated or staggered dosing regimens. The use of immiscible labeling, whether radioactivity-driven or radiotracer-driven, permits interchangeable electronic radiolabeling of single or multiple radiotracers, enabling an array of dose compilations and multiple administrations through application of the conductor in trans-vascular delivery of the electronically radiolabeled radiotracer(s).

A critical feature of trans-vascular administration of electronically radiolabeled radiotracer(s) is that systemic biodistribution is confined strictly to the duration of administration; removing the conductor stops absorption. This ensures that biodistribution is transient and ceases upon termination of administration, minimizing prolonged or off-target exposure.

Trans-vascular absorption of electronically radiolabeled radiotracer(s) establishes a framework for systemic immune modulation that unifies therapeutic effect and diagnostic monitoring into a single platform. In contrast to conventional external beam radiation therapy and total body irradiation, trans-vascular absorption of electronically radiolabeled radiotracer(s) enables the controlled distribution of ionizing energy throughout the vasculature, producing distributed biological effects that systemically extends throughout the body. By electronically radiolabeling radiotracer(s) and facilitating their absorption into the bloodstream, the approach can be adapted across oncology, infectious disease, such as HIV/AIDS, and autoimmunity to achieve targeted immune stimulation, suppression, or ablation on a systemic scale. In immune stimulation, trans-vascular absorption of electronically radiolabeled radiotracer(s) induces upregulation, proliferation, and activation of innate and adaptive immune cells, supporting oncology, infectious disease, autoimmune disease, and vaccine adjuvant applications. In immune suppression, trans-vascular absorption of electronically radiolabeled radiotracer(s) preserves immune function while targeting disease cell populations. In immune ablation, trans-vascular absorption of electronically radiolabeled radiotracer(s) induces apoptosis and necrosis of innate and adaptive immune cells, supporting pre-transplant conditioning, autoimmune disease reset, or treatment of hyperimmune states.

By introducing a low-dose of electronically radiolabeled radiotracer(s) into the bloodstream via trans-vascular absorption (e.g. less than 2 Gy), low levels of ionizing energy are systemically distributed across the vascular system and surrounding tissues. Following vascular absorption, the electronically radiolabeled radiotracer(s) produces selective stress and turnover of malignant cells, infected cells, or autoreactive immune cells. Cellular material released through this process enters circulation, where it is processed by immune cells and contributes to systemic immune activation. In this manner, systemic biodistribution of the electronically radiolabeled radiotracer(s) reshapes immune recognition and activity. In cancer, trans-vascular absorption of low-dose electronically radiolabeled radiotracer(s) promotes systemic recognition of tumor-associated material and can transform both primary and metastatic tumor sites into sources of immunogenic stimulus, effectively serving as a distributed in situ vaccination strategy. In infectious disease, the same approach enhances visibility of latent or persistent reservoirs, increasing their recognition by the immune system and supporting clearance when used alone or in combination with vaccines or antimicrobial agents. In autoimmune disease, trans-vascular absorption of low-dose electronically radiolabeled radiotracer(s) may reduce the activity of autoreactive immune populations and promote immune tolerance of self-antigens, thereby restoring balance in dysregulated immune networks.

By introducing a low (e.g. less than 2 Gy) or moderate-dose (e.g. 2-10 Gy) of electronically radiolabeled radiotracer(s) into the bloodstream via trans-vascular absorption, low or moderate levels of ionizing energy are systemically distributed throughout the vascular system, allowing immune activity to be modulated on a continuum from partial suppression to complete ablation. At moderate doses, trans-vascular absorption of electronically radiolabeled radiotracer(s) produces systemic suppression of malignant, infected, or autoreactive immune cell populations, reducing pathological activity while preserving immune function. Such suppression may be advantageous in cancer to decrease tumor-driven immune evasion, in infectious disease to weaken infected cell reservoirs, or in autoimmune disorders to attenuate autoreactive populations that drive chronic inflammation.

At higher doses (e.g. greater than 10 Gy), trans-vascular absorption of electronically radiolabeled radiotracer(s) progress from suppression to ablation, producing systemic depletion of immune and disease-associated cells. This systemic ablation can function as an immune reset, clearing pathogenic or dysregulated immune networks and preparing the body for reconstitution. In oncology, this approach can be applied prior to hematopoietic stem cell transplantation or other adoptive cell therapies, where ablation creates the necessary space and immune environment for engraftment. In infectious disease, higher-dose application can target long-lived infected immune cells, contributing to reduction to elimination of latent reservoirs. In autoimmune disease, immune ablation may be followed by stem cell therapy or controlled immune reconstitution to restore tolerance and prevent recurrence of autoreactivity. Examples of autoimmune disorders where such an approach is relevant include multiple sclerosis, systemic lupus erythematosus, rheumatoid arthritis, and type 1 diabetes.

Non-radioactive precursor(s) can be activated in vivo through trans-vascular absorption of electronically radiolabeled radiotracer(s) utilizing processes that do not require permanent activation. A non-radioactive precursor is a compound or molecule that is chemically or structurally capable of being converted into a radioactive form, but is stable and non-radioactive when administered. It serves as an inactive tracer or therapeutic before any activation occurs in the body. Non-radioactive precursors may be delivered systemically or locally via inhalation, oral ingestion, intravenous administration, transdermal absorption, intranasal administration, or localized implantation. The choice depends on target tissue and kinetics. An example of precursor activation includes electronic excitation of metal-chelate or nanoparticle complexes in which the precursor contains a stable isotope or excitable electron orbital that, when exposed to the electronic field of the electronically radiolabeled radiotracer(s) during trans-vascular absorption, undergoes transient excitation to a metastable state capable of emitting low-energy Auger electrons or secondary photons. Once trans-vascular absorption of the electronically radiolabeled radiotracer(s) cease (conductor removed from administration site), the precursor returns to its ground state, leaving the precursor chemically and radiologically inert. Another example of precursor activation involves energy-responsive molecular cages or switchable scaffolds, in which the precursor is engineered with latent binding sites that transiently trap and release electronic energy when stimulated. This process enables the precursor to be activated only during trans-vascular absorption of the electronically radiolabeled radiotracer(s), and ensures that after cessation of trans-vascular absorption, no persistent radioactivity remains in the body.

Biodistribution of electronically radiolabeled radiotracer(s) and effects can be tracked in real-time or near real-time using imaging techniques sensitive to radiotracer emissions, such as positron emission tomography (PET), single-photon emission computed tomography (SPECT), or gamma camera imaging. Imaging capture is strictly confined to the parameters of administration, as once trans-vascular absorption of the electronically radiolabeled radiotracer(s) is withdrawn, radioactivity in the body dissipates. This ensures that post-procedural radiation exposure is eliminated and that imaging can be precisely gated to the period of active administration.

Example implementations are illustrated in FIGS. 1-4, showing conductor configurations, radiotracer labeling, administration procedures, and imaging for real-time monitoring of radioactivity and effects.

As illustrated in FIG. 1, diagnostic and therapeutic protocols may be performed using a blunt-tip conductor configuration for trans-vascular absorption of electronically radiolabeled radiotracer(s). A conductive wire conductor-1 is housed within a blunt-tip needle-2 (medical grade adhesive may be used to secure and seal the wire in the blunt-tip needle and to prevent radiotracer leakage); the radiotracer-3, preferably beta or gamma-emitting, is applied via a syringe-4 directly connected with the conductor, electronically radiolabeling the radiotracer(s) upon application. The distal end-5 of the conductor is contacted with a vascular structure (e.g. superficial vein) or a surface overlying a vascular structure-6 (e.g. skin above the vein), enabling trans-vascular absorption and systemic biodistribution-7 of the electronic-radiolabeled radiotracer energy. Conductor contact is maintained for a calculated duration to deliver the desired dose in Gy. The conductor is then removed, halting systemic biodistribution-8. Cameras sensitive to radiotracer emissions, such as PET, SPECT, or gamma cameras, may be used to image and confirm dose biodistribution during administration and to monitor radioactive effects in real-time-9.

As illustrated in FIG. 2, diagnostic and therapeutic protocols may be performed using a conductive adhesive configuration for trans-vascular absorption of electronically radiolabeled radiotracer(s). A conductive adhesive tape-1 is prepared as the conductor; the radiotracer(s)-2 is applied to superabsorbent pad(s)-3 (individually, or as applicable for immiscible labeling) that is dried then pressed directly onto the conductive adhesive side of the tape-4, thereby electronically radiolabeling the radiotracer(s). The adhesive conductor is applied overlying a superficial venous site-5 for trans-vascular absorption and biodistribution of the electronic-radiolabeled radiotracer energy-6. Conductor contact is maintained for a calculated duration to deliver the desired dose in Gy. The conductor is removed, halting systemic biodistribution-7. Cameras sensitive to radiotracer emissions, such as PET, SPECT, or gamma cameras, may be used to image and confirm dose biodistribution during administration and to monitor radioactive effects in real-time-8.

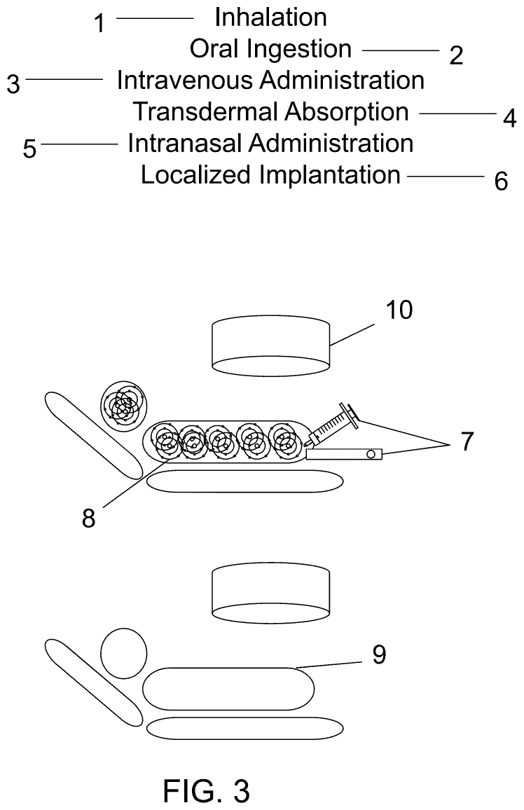

As illustrated in FIG. 3, diagnostic and therapeutic protocols may be performed by activating a non-radioactive precursor using trans-vascular absorption of electronically radiolabeled radiotracer(s) as described in FIGS. 1 and 2. The non-radioactive precursor may be introduced in the body via inhalation-1, oral ingestion-2, intravenous administration-3, transdermal absorption-4, intranasal administration-5, or localized implantation-6. The conductor(s) containing the electronically radiolabeled radiotracer(s) is contacted or applied to a vascular structure or a surface overlying a vascular structure-7, enabling trans-vascular absorption and systemic biodistribution of the electronically radiolabeled radiotracer(s)-8. Conductor contact is maintained for a calculated duration to deliver the desired dose in Gy activating the precursor. The conductor is removed, halting systemic biodistribution and activation of the precursor-9. Cameras sensitive to radiotracer emissions, such as PET, SPECT, or gamma cameras, may be used to image and confirm dose biodistribution during administration and to monitor radioactive effects in real-time-10.

As illustrated in FIG. 4, electronic radiolabeling and immiscible electronic radiolabeling of radiotracer(s) may be performed using a conductive adhesive configuration. A conductive adhesive tape-1 is prepared as the conductor; the radiotracer(s)-2 is applied to superabsorbent pad(s)-3 that is dried then pressed directly onto the conductive adhesive side of the tape, thereby electronically radiolabeling the radiotracer(s)-4. The application of two or more superabsorbent pad(s), individually hosting a radiotracer, to the conductor, without chemical conjugation, results in immiscible radiolabeling of radiotracer(s)-5.

These examples illustrate repeatable protocols that enable a skilled practitioner to implement the methods and tailor diagnostic and treatment regimens based on radiotracer selection, precursor selection, radiotracer activity, radiotracer dose rate, dosing frequency, or dose time.

A version of the invention can be fairly described as a method of labeling and administering radiotracers in which one or more radiotracer(s) are applied to a conductor made of conductive material and electronically radiolabeled by direct contact with the conductor, after which the conductor is placed in contact with a vascular structure or a surface overlying a vascular structure to enable trans-vascular absorption of the electronically radiolabeled radiotracer(s). Once absorbed, the radiotracer(s) systemically biodistribute throughout the body and may further activate non-radioactive precursor(s) in vivo. In all embodiments, systemic biodistribution of the electronically radiolabeled radiotracer(s) ceases upon removal of the conductor from the vascular structure or overlying surface. Imaging may be used to capture biodistribution and in vivo activity during trans-vascular absorption of the electronically radiolabeled radiotracer(s). The absorbed radiotracer(s) may be used for therapeutic or diagnostic purposes, including but not limited to cancer treatment, infectious disease treatment, autoimmune disease treatment, immune suppression, immune ablation, immune stimulation, immune activation, or imaging of biodistribution and immune effects. Dosimetry may be determined by measuring one or more parameters including radiotracer activity, dose rate, dose rate half-life, and dose administration time.

Although the above has been described with reference to electronic radiolabeling and administration of radiotracer(s), variations in conductor configuration, radiotracer type, dosing regimen, and imaging technique may be employed without departing from the scope of the invention, and the disclosed methods may be applied independently or synergistically to achieve both diagnostic and therapeutic outcomes.

I claim:

1. A method of labeling and administering radiotracer(s), comprising:
applying at least one radiotracer to a conductor made of conductive material;
electronically radiolabeling the radiotracer(s) by direct contact with the conductor; and
contacting the conductor to a vascular structure or to a surface overlying a vascular structure for trans-vascularly absorbing the electronically radiolabeled radiotracer(s).

2. A method of labeling and administering radiotracer(s), comprising:
applying at least one radiotracer to a conductor made of conductive material;
electronically radiolabeling the radiotracer(s) by direct contact with the conductor;
contacting the conductor to a vascular structure or to a surface overlying a vascular structure for trans-vascularly absorbing the electronically radiolabeled radiotracer(s); and
systemically biodistributing the electronically radiolabeled radiotracer(s).

3. A method of labeling and administering radiotracer(s), comprising:
applying at least one radiotracer to a conductor made of conductive material;
electronically radiolabeling the radiotracer(s) by direct contact with the conductor;
contacting the conductor to a vascular structure or to a surface overlying a vascular structure for trans-vascularly absorbing the electronically radiolabeled radiotracer(s);
systemically biodistributing the electronically radiolabeled radiotracer(s); and
activating non-radioactive precursor(s) in the body.

4. The method of claim 2, wherein trans-vascularly absorbing the electronically radiolabeled radiotracer(s) enables immunomodulatory activity in the body.

5. The method of claim 3, wherein trans-vascularly absorbing the electronically radiolabeled radiotracer(s) enables immunomodulatory activity in the body.

6. The method of claim 2, wherein trans-vascularly absorbing the electronically radiolabeled radiotracer(s) enables immunomodulatory activity in the body as immune suppression.

7. The method of claim 3, wherein trans-vascularly absorbing the electronically radiolabeled radiotracer(s) enables immunomodulatory activity in the body as immune suppression.

8. The method of claim 2, wherein trans-vascularly absorbing the electronically radiolabeled radiotracer(s) enables immunomodulatory activity in the body as immune ablation.

9. The method of claim 3, wherein trans-vascularly absorbing the electronically radiolabeled radiotracer(s) enables immunomodulatory activity in the body as immune ablation.

10. The method of claim 2, wherein trans-vascularly absorbing the electronically radiolabeled radiotracer(s) enables immunomodulatory activity in the body as immune stimulation or immune activation.

11. The method of claim 3, wherein trans-vascularly absorbing the electronically radiolabeled radiotracer(s) enables immunomodulatory activity in the body as immune stimulation or immune activation.

12. The method of claim 2, further comprising ceasing systemic biodistribution of the electronically radiolabeled radiotracer(s) upon removal of the conductor from the vascular structure or surface overlying the vascular structure.

13. The method of claim 3, further comprising ceasing systemic biodistribution of the electronically radiolabeled radiotracer(s) and activation of the precursor upon removal of the conductor from the vascular structure or surface overlying the vascular structure.

14. The method of claim 2, further comprising capturing imaging data of the electronically radiolabeled radiotracer biodistribution and activity in the body during trans-vascularly absorbing the electronically radiolabeled radiotracer(s).

15. The method of claim 3, further comprising capturing imaging data of the electronically radiolabeled radiotracer biodistribution and activity in the body during trans-vascularly absorbing the electronically radiolabeled radiotracer(s).

16. The method of claim 2, wherein trans-vascularly absorbing the electronically radiolabeled radiotracer(s) is cancer treatment.

17. The method of claim 3, wherein trans-vascularly absorbing the electronically radiolabeled radiotracer(s) is cancer treatment.

18. The method of claim 2, wherein trans-vascularly absorbing the electronically radiolabeled radiotracer(s) is infectious disease treatment.

19. The method of claim 3, wherein trans-vascularly absorbing the electronically radiolabeled radiotracer(s) is infectious disease treatment.

20. The method of claim 2, wherein trans-vascularly absorbing the electronically radiolabeled radiotracer(s) is autoimmune disease treatment.

21. The method of claim 3, wherein trans-vascularly absorbing the electronically radiolabeled radiotracer(s) is autoimmune disease treatment.

22. The method of claim 2, wherein trans-vascularly absorbing the electronically radiolabeled radiotracer(s) is for diagnostic purposes.

23. The method of claim 3, wherein trans-vascularly absorbing the electronically radiolabeled radiotracer(s) is for diagnostic purposes.

24. The method of claim 2, wherein trans-vascularly absorbing the electronically radiolabeled radiotracer(s) is for therapeutic purposes.

25. The method of claim 3, wherein trans-vascularly absorbing the electronically radiolabeled radiotracer(s) is for therapeutic purposes.

26. The method of claim 1, further comprising determining an absorbed dose of the electronically radiolabeled radiotracer(s) based on at least one of: radiotracer activity, dose rate, dose rate half-life, and dose time.
